(19) United States
(12) Patent Application Publication
(10) Pub. No.: US 2007/0293552 A1
(43) Pub. Date: Dec. 20, 2007

(54) ANTIHYPERTENSIVE THERAPY METHOD

(76) Inventor: Richard J. Gorczynski, Westminster, CO (US)

Correspondence Address: HARNESS, DICKEY, & PIERCE, P.L.C, 7700 BONHOMME, STE 400, ST. LOUIS, MO, 63105, US

(21) Appl. No.: 11/761,499

(22) Filed: Jun. 12, 2007

Related U.S. Application Data
(60) Provisional application No. 60/813,966, filed on Jun. 15, 2006.

(51) Int. Cl. A61K 31/42

(52) U.S. Cl. 514/378

(57) ABSTRACT

Methods and therapeutic combinations are provided for lowering blood pressure in a subject exhibiting resistance to a baseline antihypertensive therapy with one or more drugs, or a subject having diabetes and/or chronic kidney disease. A method of the invention comprises administering, in some embodiments adjunctively with a modified baseline therapy, a compound of formula (I) as defined herein. A therapeutic combination of the invention comprises a compound of formula (I); at least one diuretic; and at least one antihypertensive drug selected from ACE inhibitors, angiotensin II receptor blockers, beta-adrenergic receptor blockers and calcium channel blockers; wherein the at least one diuretic and/or the at least one antihypertensive drug are present at substantially less than a full dose.

This application claims the benefit of U.S. provisional application Ser. No. 60/813,966, filed Jun. 15, 2006, incorporated in its entirety herein by reference.

### FIELD OF THE INVENTION

The present invention relates to methods and therapeutic combinations useful for lowering blood pressure.

### BACKGROUND OF THE INVENTION

Blood pressure control can often be achieved by antihypertensive therapy with one or more drugs. Despite a wide range of drugs available for antihypertensive therapy, a segment of the patient population continues to exhibit resistance to a baseline antihypertensive therapy with one or more drugs. A particularly challenging patient population has resistant hypertension. Resistant hypertension is defined by the Seventh Report of the Joint National Committee on Prevention, Detection, Evaluation, and Treatment of High Blood Pressure (JNC 7; Chobanian et al. (2003) Hypertension 42:1206-1252) as a failure to achieve goal blood pressure in patients who are adhering to full doses of an appropriate three-drug regimen that includes a diuretic. Further, resistant hypertension is diagnosed by many physicians on the basis of a patient's resistance to adequate, but less than full, doses of an appropriate three-drug regimen because of the risk or occurrence of adverse events associated with full doses. The terms “adequate” and “full” in the present context are defined below.

According to JNC 7, a goal of systolic blood pressure (SBP) <140 mmHg and diastolic blood pressure (DBP) <90 mmHg is recommended for patients with hypertension and no other serious conditions. For patients with serious or compelling conditions such as diabetes and chronic kidney disease, JNC 7 recommends a goal of SBP <130 mmHg and DBP <80 mmHg. Despite intensive, multi-drug therapy, only about 50% of patients with diabetes or chronic kidney disease reach traditional blood pressure goals, with even fewer reaching the more stringent goals now recommended by JNC 7. Thus, resistant hypertension is particularly acute for segments of the population which exhibit diabetes or chronic kidney disease.

It should be noted that the British Hypertensive Society (BHD-IV; J. Human Hypertens. (2004) 18:139-185), the European Society of Hypertension/European Society of Cardiology (ESH/ESC; J. Hypertens. (2003) 21:1011-1053), and the World Health Organization/International Society of Hypertension (WHO/ISH; J. Hypertens. (2003) 21:1983-1992) guidelines propose similar but not identical blood pressure goals for non-diabetic and diabetic patients.

Use of various selective endothelin-A (ET_{A}) receptor antagonists to treat moderate hypertension has been proposed or tested in clinical trials. Endothelin (more particularly the ET-1 isoform thereof) is a small peptide hormone that is believed to play a critical role in control of blood flow and cell growth. Elevated endothelin blood levels are associated with several cardiovascular disease conditions, including pulmonary arterial hypertension, chronic renal disease, coronary artery disease, hypertension, and chronic heart failure. Endothelin is a potent vasoconstrictor, triggering contraction through endothelin-receptor mediated signaling pathways. While antagonism of the ET_{A} receptor is known to reduce endothelin-mediated vasoconstriction, antagonism of the endothelin-B (ET_{B}) receptor can block clearance of ET-1 from the circulatory system, exacerbating its hypertensive effect.

Wu et al. (2004) J. Med. Chem. 47, 1969-1986 presented comparative information on a range of compounds having varying degrees of affinity and selectivity for ET_{A} and having a general formula (I) that can be represented: 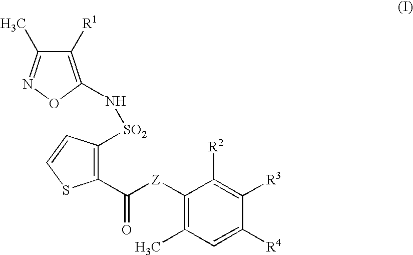
where, in certain of the compounds listed:
R¹ is halo or C₁₋₃ alkyl;
R² is hydrogen, C₁₋₃ alkyl, (C₁₋₃ alkyl)carbonyl, (C₃₋₆ cycloalkyl)carbonyl, cyano, halo, aminocarbonyl, di(C₁₋₃ alkyl)aminocarbonyl, (C₁₋₃ alkyl)sulfonyl, oxazol-2-yl or benzoyl;
R³ and R⁴ are independently hydrogen or C₁₋₃ alkyl, or together form a methylenedioxy bridge; and
Z is CH₂ or NH.
   Among the compounds mentioned by Wu et al. are:
sitaxsentan (N-(4-chloro-3-methylisoxazol-5-yl)-2-(2-(6-methyl-3,4-methylenedioxy-1-yl)acetyl)thiophen-3-sulfonamide), in which R¹ is chloro, R² is hydrogen, R³ and R⁴ form a methylenedioxy bridge, and Z is CH₂; and
TBC3711 (N-(2-acetyl-4,6-dimethylphenyl)-3-(3,4-dimethylisoxazol-5-ylsulfamoyl)-thiophene-2-carboxamide), in which R¹ is methyl, R² is acetyl, R³ is hydrogen, R⁴ is methyl, and Z is NH.
   Wu et al. reported an ET_{A} binding affinity (IC₅₀) of 1.4±0.5 nM for sitaxsentan and 0.08±0.02 nM for TBC3711, and an ET_{A}/ET_{B} selectivity factor of 6.5×10³ for sitaxsentan and 441×10³ for TBC3711.

A clinical trial to test a selective ET_{A} receptor antagonist of a different chemical class, namely darusentan, for treatment of resistant hypertension was proposed in a Myogen, Inc. news release dated Jul. 15, 2004 (http://investor.myogen.com/phoenix.zhtml?c=135160&p=iro1-newsArticle&ID=759464&highlight=), later reported in Heart Disease Weekly, Aug. 15, 2004, p. 113.

New methods for lowering blood pressure continue to be much needed in the art. Such new methods would be of value if they afford one or more benefits over existing therapies, for example one or more benefits as mentioned below, it being understood that recitation of any particular benefit sought does not limit the present invention to embodiments exhibiting that benefit.

Improved drug therapies for treatment of patients exhibiting resistance to a baseline antihypertensive therapy with one or more drugs, and especially patients having resistant hypertension, would be highly desirable. Resistant hypertension is increasing in prevalence due to a variety of contributing factors including an aging population, obesity, patient noncompliance, and the effects of target-organ disease. Thus, a focus of current treatment of resistant hypertension is to identify and eliminate contributing factors. Despite reducing contributing factors, a substantial proportion of patients with resistant hypertension fail to achieve blood pressure goals.

Furthermore, a baseline antihypertensive therapy, whether or not a patient exhibits resistance thereto, can have undesirable, in some cases clinically unacceptable or even dangerous, adverse side effects, especially when administered at a full dose of each constituent drug in the therapy.

Thus of particular benefit would be an effective drug therapy for treatment of patients exhibiting resistance to a baseline antihypertensive therapy with one or more drugs, and especially patients having resistant hypertension, wherein the baseline therapy can be modified in a manner that results in a reduced risk or incidence of adverse events.

Ambulatory blood pressure in most patients exhibits a 24-hour cycle wherein both systolic and diastolic blood pressures are typically higher during the day than at night. Any beneficial change in a patient's 24-hour pattern of ambulatory systolic and/or diastolic blood pressure would be desirable where the patient exhibits resistance to a baseline antihypertensive therapy with one or more drugs, and especially where the patient has resistant hypertension.

In patients having resistant hypertension, improved renal and/or cardiovascular function, including prevention of cardiovascular adverse events, would be of great benefit.

As mentioned above, control of hypertension is especially critical for patients having compelling conditions such as diabetes and/or chronic kidney disease. A new drug therapy for lowering blood pressure in such patients is therefore another important desideratum.

### SUMMARY OF THE INVENTION

In various embodiments of the present invention, one or more of the benefits sought above, and/or further benefits that will become apparent on reading the detailed description that follows, can be realized.

Accordingly, there is now provided a method for lowering blood pressure in a subject exhibiting resistance to a baseline antihypertensive therapy with one or more drugs. The method comprises administering to the subject, adjunctively with the baseline therapy, a compound of formula (I) 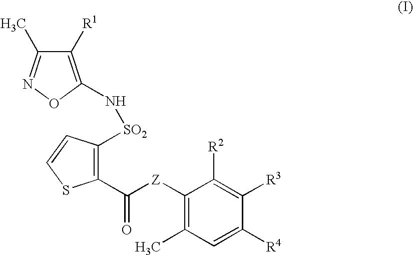
or a pharmaceutically acceptable salt thereof, where:
R¹ is halo or C₁₋₃ alkyl;
R² is hydrogen, C₁₋₃ alkyl, (C₁₋₃ alkyl)carbonyl, (C₃₋₆ cycloalkyl)carbonyl, cyano, halo, aminocarbonyl, di(C₁₋₃ alkyl)aminocarbonyl, (C₁₋₃ alkyl)sulfonyl, oxazol-2-yl or benzoyl;
R³ and R⁴ are independently hydrogen or C₁₋₃ alkyl, or together form a methylenedioxy bridge; and
Z is CH₂or NH;
   wherein
(a) the baseline therapy is modified by dose reduction or elimination of at least one of said drugs;
(b) the baseline therapy is modified in a manner that results in a reduced risk or incidence of adverse events; and/or
(c) a beneficial change in the subject's 24-hour pattern of systolic and/or diastolic blood pressure is obtained.
   A closely related embodiment provides use of a compound of formula (I) in preparation of a medicament for adjunctive administration to lower blood pressure in a subject exhibiting resistance to a baseline antihypertensive therapy with one or more drugs; said adjunctive administration being with the baseline therapy, which is modified (a) by dose reduction or elimination of at least one of said drugs and/or (b) in a manner that results in a reduced risk or incidence of adverse events.

There is further provided a method for lowering blood pressure in a subject having diabetes and/or chronic kidney disease. The method comprises administering a compound of formula (I) above or a pharmaceutically acceptable salt thereof to the subject. A closely related embodiment provides use of a compound of formula (I) in preparation of a medicament for lowering blood pressure in a subject having diabetes and/or chronic kidney disease.

There is still further provided a method for providing a beneficial effect on renal and/or cardiovascular function in a subject having resistant hypertension. The method comprises administering a compound of formula (I) above or a pharmaceutically acceptable salt thereof to the subject. A closely related embodiment provides use of a compound of formula (I) in preparation of a medicament for providing a beneficial effect on renal and/or cardiovascular function in a subject having resistant hypertension.

There is still further provided a therapeutic combination comprising a compound of formula (I) above or a pharmaceutically acceptable salt thereof, at least one diuretic, and at least one antihypertensive drug selected from ACE inhibitors, angiotensin II receptor blockers, beta-adrenergic receptor blockers and calcium channel blockers, wherein the at least one diuretic and/or the at least one antihypertensive drug are present at substantially less than a full dose.

In the above combination and in all the above methods, the compound of formula (I) is, in a first embodiment, sitaxsentan (N-(4-chloro-3-methylisoxazol-5-yl)-2-(2-(6-methyl-3,4-methylenedioxy-1-yl)acetyl)thiophen-3-sulfonamide), or, in a second embodiment, TBC3711 (N-(2-acetyl-4,6-dimethylphenyl)-3-(3,4-dimethylisoxazol-5-ylsulfamoyl)-thiophene-2-carboxamide).

Other embodiments, including particular aspects of the embodiments summarized above, will be evident from the detailed description that follows.

### DETAILED DESCRIPTION

Initiation of patient enrollment in a study to evaluate once-daily oral administration of TBC3711 in patients with resistant hypertension was announced in an Encysive Pharmaceuticals news release dated Jan. 9, 2006 (http://ir.encysive.com/ireye/ir_site.zhtml?ticker=ency&script=410&layout=6&item_id=801805). More details of this proposed study, including inclusion and exclusion criteria, have been posted (http://www.clinicaltrials.gov/ct/show/NCT00272961?order=1). These disclosures are incorporated herein by reference but no admission is made as to their status as prior art or otherwise with respect to the present invention.

In various aspects of the invention, methods are provided for lowering blood pressure in subjects exhibiting resistance to a baseline antihypertensive therapy with one or more drugs, including subjects having resistant hypertension as in the proposed TBC3711 study referenced above. “Resistant hypertension” is to be understood herein as resistance at least to adequate doses of an appropriate three-drug antihypertensive regimen that includes a diuretic. Typically resistant hypertension is diagnosed clinically. According to some of these methods, any one or more measures of blood pressure can be lowered, including SBP and/or DBP as determined, for example, by sphygmomanometry. According to certain embodiments, as indicated with particularity hereinbelow, one or more particular measures of blood pressure are specified.

SBP and/or DBP can be measured, for example, in a sitting or ambulatory subject.

A “trough sitting” SBP or DBP is measured at a time point when serum concentration of a drug or drugs administered according to a method of the invention is expected to be at or close to its lowest in a treatment cycle, typically just before administration of a further dose. Illustratively, where the drug or drugs are administered once a day at a particular time, for example around 8 am, trough sitting SBP or DBP can be measured at that time, immediately before the daily administration. It is generally preferred to measure trough sitting SBP or DBP at around the same time of day for each such measurement, to minimize variation due to the natural 24-hour blood pressure cycle.

The course of the 24-hour blood pressure cycle is most conveniently tracked by ambulatory blood pressure (ABP) monitoring.

A “24-hour ambulatory” SBP or DBP is an average of measurements taken repeatedly in the course of a 24-hour period, in an ambulatory subject.

A “maximum diurnal” SBP or DBP is a measure of highest SBP or DBP recorded in a 24-hour period, for example by ABP monitoring, and often reflects the peak of the natural 24-hour blood pressure cycle, typically occurring in the morning, for example between about 5 am and about 11 am. Commonly, a second peak occurs in the evening, for example between about 5 pm and 10 pm. Such a bimodal waveform 24-hour ABP pattern may be especially characteristic of resistant hypertension.

A common feature of resistant hypertension is a night-time (defined herein as 2200 (10 pm) to 0600 (6 am)) mean systolic ABP that is no lower, or lower by a margin of less than about 10%, than the daytime (defined herein as 0600 to 2200) mean systolic ABP. The parameter herein termed “day/night ABP ratio” expressed as a percentage is calculated from daytime and night-time mean systolic ABP using the formula
(daytime mean−night-time mean)/daytime mean×100.
A 24-hour ABP pattern having a day/night ABP ratio of less than about 10% is sometimes referred to as a “non-dipping ABP”.

The term “subject” refers to a warm-blooded animal, generally a mammal such as, for example, a primate, including a human. In one embodiment the subject is a human, for example a patient having clinically diagnosed hypertension.

As indicated above, the subject receiving blood pressure lowering (antihypertensive) therapy according to a method of the invention can be a subject exhibiting resistance to a baseline antihypertensive therapy with one or more drugs. A “baseline antihypertensive therapy” herein means a therapeutic regimen comprising administration of one or more drugs, with an objective (which can be the primary objective or a secondary objective of the regimen) of lowering blood pressure in a hypertensive subject. Each drug according to the regimen is administered at least at a dose considered by an attending physician to be adequate for treatment of hypertension, taking into account the particular subject's medical condition and tolerance for the drug without unacceptable adverse side-effects. An “adequate” dose as prescribed by the physician can be less than or equal to a full dose of the drug. A “full” dose is the lowest of (a) the highest dose of the drug labeled for a hypertension indication; (b) the highest usual dose of the drug prescribed according to JNC 7, BHD-IV, ESH/ESC or WHO/ISH guidelines; or (c) the highest tolerated dose of the drug in the particular subject.

A baseline antihypertensive therapy illustratively comprises administering one or more diuretics and/or one or more antihypertensive drugs selected from (a) angiotensin converting enzyme (ACE) inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) vasopeptidase inhibitors, (i) neutral endopeptidase (NEP) inhibitors, (j) prostanoids, (k) phosphodiesterase type 5 (PDE5) inhibitors, (l) nitrosylated compounds, (m) oral nitrates and (n) inhibitors of renin activity or release. Optionally drugs of still further classes can be included in the baseline therapy, for example to address secondary conditions occurring in a hypertensive subject or side-effects of one or more of the diuretic or antihypertensive drugs.

A subject who is “resistant” to a baseline antihypertensive therapy is one in whom hypertension is failing to respond adequately or at all to the baseline therapy. Typically, the subject receiving the baseline therapy is failing to reach an established blood pressure goal, as set forth for U.S. patients, for example, in JNC 7 or comparable standards in other countries (e.g., BHD-IV, ESH/ESC or WHO/ISH guidelines). Illustratively, the JNC 7 goal for SBP is <140 mmHg and for DBP <90 mmHg, or for a subject having a complicating condition such as diabetes and/or chronic kidney disease, <130 mmHg SBP and <80 mmHg DBP.

The compound administered according to the methods of the present invention is an endothelin receptor antagonist, preferably a selective ET_{A} receptor antagonist, for example one having an IC₅₀ for ET_{A} not greater than about 10 nM, e.g., not greater than about 5 nM or not greater than about 2 nM, and/or an ET_{A}/ET_{B} selectivity factor of at least about 100, e.g., at least about 500 or at least about 1000.

The compound is a member of a class of endothelin receptor antagonists having the formula (II)
Ar²—SO₂—NH—Ar¹ (II)
or a pharmaceutically acceptable salt thereof, where:
Ar¹ is a monocyclic or polycyclic heteroaryl moiety, unsubstituted or substituted with one or more substituents independently selected from amino, halo, alkyl, acyl, aryl, heteroaryl, alkoxyalkyl, alkylamino, alkylthio, arylcarbonyl, aryloxy, arylamino, arylthio, haloalkyl, haloaryl and carbonyl groups;
Ar² is a moiety 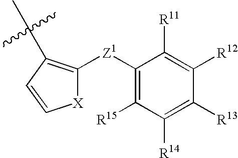
   where:
   Z¹ is a linking group (CH₂)ₘC(O)(CH₂)ᵣ, (CH₂)ₘC(O)NH(CH₂)ᵣ, CH(OH)(CH₂)ᵣ, (CH)ₘ(CH═CH)(CH₂)ᵣ, (CH₂)ₘC(O)(CH₂)ₛNH(CH₂)ᵣ, C═N(OH)(CH₂)ᵣ, (CH₂)ₘC(O)(CH═CH)ₛNH(CH₂)ᵣ, CH(CH₃)C(O)(CH₂)ᵣ, CH(CH₃)C(O)(CH₂)ₘ(CH═CH)(CH₂)ᵣ, (CH₂)ᵣ, (CH₂)ᵣ(O) or C(O)O, in which m, s, and r are each independently an integer from zero to 6;
   R¹¹, R¹², R¹³, R¹⁴ and R¹⁵ are independently selected from hydrogen, hydroxy, NHR⁸, CONR⁸R⁹, nitro, cyano, halo, alkyl, alkenyl, alkynyl, aryl, arylalkyl, heteroaryl, alkoxy, alkenyloxy, alkylamino, alkenylamino, alkylthio, alkenylthio, haloalkyl, alkylsulfinyl, alkenylsulfinyl, alkylsulfonyl, alkenylsulfonyl, alkylcarbonyl, alkoxycarbonyl, alkylaminocarbonyl, arylaminocarbonyl, aminocarbonyl, (alkylaminocarbonyl)alkyl, carboxyl, carboxyalkyl, carboxyalkenyl, alkylsulfonylaminoalkyl, cyanoalkyl, acetyl, acetoxyalkyl, hydroxyalkyl, alkoxyalkoxy, acetoxyalkoxy, hydroxyalkoxy and formyl groups; or at least two of R¹¹, R¹², R¹³, R¹⁴ and R¹⁵, which substitute adjacent carbons on the ring, together form an alkylenedioxy, alkylenethioxyoxy or alkylenedithioxy bridge, which is optionally substituted with halo, alkyl, alkoxy or haloalkyl groups, and the others of R¹¹, R¹², R¹³, R¹⁴ and R¹⁵ are independently selected as above; where, in NHR⁸ and CONR⁸R⁹ groups, R⁸ and R⁹ are independently selected from hydrogen, alkyl, alkenyl, alkynyl, aryl, haloalkyl, alkylaryl, heterocyclyl, arylalkyl, arylalkoxy, alkoxy, aryloxy, cycloalkyl, cycloalkenyl and cycloalkynyl; and
   X is O, N or S;
      in which alkyl or acyl portions comprise 1 to about 12, for example 1 to about 6, carbon atoms and alkenyl or alkynyl portions comprise 2 to about 12, for example 2 to about 6, carbon atoms, and can form straight or branched chains; and cycloalkyl, cycloalkenyl and cycloalkynyl portions comprise 3 to about 12, for example 3 to about 6, carbon atoms.

In a compound of formula (II) where Z¹ is (CH₂)ₘC(O)NH(CH₂)ᵣ, at least two of R¹¹, R¹², R¹³, R¹⁴ and R¹⁵ are other than hydrogen.

Illustratively, the compound administered can have formula (II) where
Ar¹ is a monocyclic 5-membered ring having 1 to 3 heteroatoms in the ring, at least one of which is N, and is unsubstituted or substituted with one or more halo or alkyl, (e.g., C₁₋₃ alkyl) substituents;
Z¹ is (CH₂)ₘC(O)(CH₂)ᵣ, for example where m is zero and r is 1, C(O)CH₂; or (CH₂)ₘC(O)NH(CH₂)ᵣ, for example where m and r are each zero, C(O)NH; and
X is S.

Such compounds and processes for their preparation are disclosed, for example in U.S. Pat. No. 6,248,767 to Blok et al., incorporated herein by reference.

The invention is described herein with particular reference to a compound of formula (I) 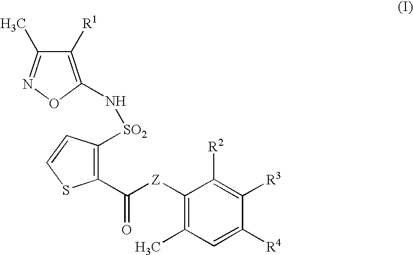
or a pharmaceutically acceptable salt thereof, where:
R¹ is halo or C₁₋₃ alkyl;
R² is hydrogen, C₁₋₃ alkyl, (C₁₋₃ alkyl)carbonyl, (C₃₋₆ cycloalkyl)carbonyl, cyano, halo, aminocarbonyl, di(C₁₋₃ alkyl)aminocarbonyl, (C₁₋₃ alkyl)sulfonyl, oxazol-2-yl or benzoyl;
R³ and R⁴ are independently hydrogen or C₁₋₃ alkyl, or together form a methylenedioxy bridge; and
Z is CH₂ or NH.

In one embodiment, in the compound of formula (I), R¹ is C₁₋₃ alkyl; R² is hydrogen or C₁₋₃ alkyl; R³ and R⁴ are independently hydrogen or C₁₋₃ alkyl, or together form a methylenedioxy bridge; and Z is CH₂ or NH.

According to this embodiment, the compound can illustratively be sitaxsentan or TBC3711. Sitaxsentan (N-(4-chloro-3-methylisoxazol-5-yl)-2-(2-(6-methyl-3,4-methylenedioxy-1-yl)acetyl)thiophen-3-sulfonamide) has formula (I) where R¹ is chloro, R² is hydrogen, R³ and R⁴ form a methylenedioxy bridge, and Z is CH₂, and can be represented as 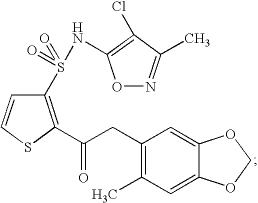
and TBC3711 (N-(2-acetyl-4,6-dimethylphenyl)-3-(3,4-dimethylisoxazol-5-ylsulfamoyl)-thiophene-2-carboxamide) has formula (I) where R¹ is methyl, R² is acetyl, R³ is hydrogen, R⁴ is methyl, and Z is NH, and can be represented as

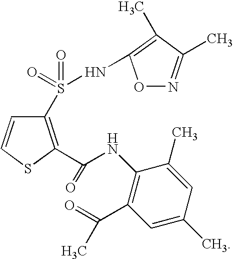

Processes for preparing the above compounds are found, for example, in Wu et al. (2004) J. Med. Chem. 47, 1969-1986, incorporated by reference herein.

The invention is not limited to any route of administration of the compound of formula (I), so long as the route selected results in effective delivery of the drug so that a benefit is obtainable. Thus administration of the compound of formula (I) can illustratively be parenteral (e.g., intravenous, intraperitoneal, subcutaneous or intradermal), transdermal, transmucosal (e.g., buccal, sublingual or intranasal), intraocular, intrapulmonary (e.g., by inhalation) or rectal. Most conveniently for the majority of subjects, however, the compound of formula (I) is administered orally, i.e., per os (p.o.). Any suitable orally deliverable dosage form can be used for the compound of formula (I), including without limitation tablets, capsules (solid- or liquid-filled), powders, granules, syrups and other liquids, etc.

For oral administration, any dose of the compound of formula (I) that is therapeutically effective, up to a maximum that is tolerated by the subject without unacceptable adverse side effects, can be administered. Illustratively, in the case of TBC3711, such a dose for most subjects is likely to be about 1 to about 600 mg/day, for example about 10 to about 200 mg/day. Higher or lower doses can be useful in specific circumstances.

The prescribed daily dosage amount can be administered in any suitable number of individual doses, for example four times, three times, twice or once a day. With a dosage form having appropriate controlled release properties, a lower frequency of administration may be possible, for example once every two days, once a week, etc.

Most antihypertensive medicines are suitable for once a day administration, and, where the compound of formula (I) is likewise suitable for once a day administration, it is generally most convenient to administer the compound of formula (I) once a day, for example orally in a dose as indicated above for TBC3711 or, for another compound, a dose providing equivalent therapeutic efficacy. This is particularly true in those embodiments of the invention wherein the compound of formula (I) is administered adjunctively with other antihypertensive drugs, for example in a modified baseline therapy.

Subjects resistant to a baseline antihypertensive therapy, especially such a therapy involving a plurality of drugs, clearly represent a very challenging population for treatment. Typically in such subjects, increasing dosages of the baseline therapy are not an option because of resulting adverse side effects; furthermore this approach is often ineffective in providing a desired lowering of blood pressure. Accordingly, in various embodiments of the present invention, a method for lowering blood pressure in a subject exhibiting resistance to a baseline antihypertensive therapy with one or more drugs comprises administering to the subject, adjunctively with the baseline therapy, a compound of formula (I) above or a pharmaceutically acceptable salt thereof, wherein the baseline therapy is modified (a) by dose reduction or elimination of at least one of said drugs; and/or (b) in a manner that results in a reduced risk or incidence of adverse events. “Modified” herein means by comparison with the baseline therapy to which the subject has exhibited resistance.

In one such embodiment, the adjunctively administered baseline therapy is modified by dose reduction or elimination of at least one of said drugs.

Dose reduction according to the present embodiment can comprise any degree of dose reduction resulting in a dose that is substantially less than a full dose as defined above. In many situations the reduced dose can be substantially less than the dose of the drug previously used in the baseline therapy to which the subject has exhibited resistance, even where such dose was less than a full dose. For example, without limitation, a dose of one or more drugs in the baseline therapy can be reduced by at least about 10%, at least about 25%, or at least about 50%, and can be reduced by as much as about 75% or more, by comparison with a full dose. Alternatively or in addition, at least one drug in the baseline therapy can be eliminated in its entirety.

Dose reduction or elimination of a baseline therapy drug permitted by use of a compound of formula (I) can result in a reduced risk or incidence of adverse events by comparison with the baseline therapy alone without such dose reduction or elimination. Accordingly, in one aspect of the present method, a reduced risk or incidence of adverse events is obtained by comparison with the baseline therapy alone without said dose reduction or elimination.

Particularly when used at a full dose, many baseline antihypertensive therapy drugs can have undesirable, in some cases clinically unacceptable or even dangerous, adverse side effects. For example, especially at full doses, potassium-sparing diuretic drugs can be associated with increased risk of hyperkalemia and related disorders. Overuse of loop diuretics can cause depletion of sodium resulting in hyponatremia and/or extracellular fluid volume depletion associated with hypotension, reduced GFR (glomerular filtration rate), circulatory collapse, and thromboembolic episodes. Further, loop diuretics can cause ototoxicity that results in tinnitus, hearing impairment, deafness and/or vertigo. Thiazide diuretics, similarly to loop diuretics, can have adverse effects related to abnormalities of fluid and electrolyte balance. Such adverse events include extracellular volume depletion, hypotension, hypokalemia, hyponatremia, hypochloremia, metabolic alkalosis, hypomagnesemia, hypercalcemia and hyperuricemia. Thiazide diuretics can also decrease glucose tolerance, and increase plasma levels of LDL (low density lipoprotein) cholesterol, total cholesterol, and total triglycerides.

ACE inhibitors are associated with cough and increased risk of angioedema. Beta-adrenergic receptor blockers are associated with increased risk of bronchospasm, bradycardia, heart block, excess negative inotropic effect, peripheral arterial insufficiency and sometimes male impotence. Calcium channel blockers are associated with increased risk of lower limb edema. Aldosterone receptor antagonists can cause gynecomastia. Further information on adverse events associated with antihypertensive drugs can be found, for example, in standard reference works such as Goodman & Gilman's The Pharmaceutical Basis of Therapeutics, 13th ed. (Brunton et al., eds. (2006), New York: McGraw Hill).

In the method of the present embodiment, the compound of formula (I) is illustratively administered adjunctively with a modified baseline therapy that comprises administration of one or more diuretics and/or one or more antihypertensive drugs selected from (a) ACE inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) vasopeptidase inhibitors, (i) NEP inhibitors, (j) prostanoids, (k) PDE5 inhibitors, (l) nitrosylated compounds, (m) oral nitrates and (n) inhibitors of renin activity and release.

“Adjunctive” administration of a compound of formula (I) herein means that the compound of formula (I) is administered concomitantly with one or more additional drugs, in the present instance one or more drugs constituting a modified baseline therapy. For example, a compound of formula (I) can be administered adjunctively with an adequate to full dose of one or more of the drugs in the baseline therapy, while the other one or more drugs in the baseline therapy are administered at reduced dose or eliminated.

The method of the present embodiment, in common with methods of other embodiments described herein, can be especially beneficial where the subject has resistant hypertension. By definition herein, in general accordance with JNC 7, such a subject exhibits resistance to an antihypertensive regimen of at least three drugs including a diuretic. In one embodiment, the subject having resistant hypertension exhibits resistance to a baseline antihypertensive therapy that comprises at least the following:
(1) one or more diuretics; and
(2) two or more antihypertensive drugs, selected from at least two of the following classes:
   (a) ACE inhibitors and angiotensin II receptor blockers;
   (b) beta-adrenergic receptor blockers; and
   (c) calcium channel blockers.

In some cases, the subject is resistant to an even more comprehensive baseline therapy, further comprising, for example, one or more direct vasodilators, alpha-1-adrenergic blockers, central alpha-2-adrenergic agonists or other centrally acting antihypertensive drugs, aldosterone receptor antagonists, vasopeptidase inhibitors, NEP inhibitors, prostanoids, PDE5 inhibitors, nitrosylated compounds, oral nitrates and/or inhibitors of renin activity or release.

In one aspect of the present embodiment, the compound of formula (I) is administered at a dose and frequency effective, in combination with the modified baseline therapy, to provide a reduction of at least about 3 mmHg in one or more blood pressure parameters selected from trough sitting SBP, trough sitting DBP, 24-hour ambulatory SBP, 24-hour ambulatory DBP, maximum diurnal SBP and maximum diurnal DBP.

In a particular aspect, the subject has resistant systolic hypertension, and the dose and frequency of administration of the compound of formula (I) is effective in combination with the modified baseline therapy to provide a reduction of at least about 3 mmHg in one or more of trough sitting, 24-hour ambulatory and maximum diurnal SBP.

In a further particular aspect, the at least about 3 mmHg reduction is observed in trough sitting SBP, and at least comparable reductions can be, but are not necessarily, observable in 24-hour ambulatory and/or maximum diurnal SBP. In some cases the method is effective to provide a greater reduction in trough sitting SBP, for example at least about 5 mmHg, at least about 7 mmHg or at least about 10 mmHg.

The present method can increase the likelihood of a subject achieving SBP goal, for example a JNC 7, BHD-IV, ESH/ESC or WHO/ISH goal for SBP. Thus in a still further particular aspect, a JNC 7 goal for SBP is achieved, for example a trough sitting or 24-hour ambulatory SBP of <140 mmHg or, in the case of a subject with diabetes or chronic kidney disease, <130 mmHg.

In another particular aspect, the subject has resistant diastolic hypertension, and the dose and frequency of administration of the compound of formula (I) is effective in combination with the modified baseline therapy to provide a reduction of at least about 3 mmHg in one or more of trough sitting, 24-hour ambulatory and maximum diurnal DBP.

In a further particular aspect, the at least about 3 mmHg reduction is observed in trough sitting DBP, and at least comparable reductions can be, but are not necessarily, observable in 24-hour ambulatory and/or maximum diurnal DBP. In some cases the method is effective to provide a greater reduction in trough sitting DBP, for example at least about 5 mmHg, at least about 7 mmHg or at least about 10 mmHg.

The present method can increase the likelihood of a subject achieving DBP goal, for example a JNC 7, BHD-IV, ESH/ESC or WHO/ISH goal for DBP. Thus in a still further particular aspect, a JNC 7 goal for DBP is achieved, for example a trough sitting or 24-hour ambulatory DBP of <90 mmHg or, in the case of a subject with diabetes or chronic kidney disease, <80 mmHg.

In yet another aspect, the method of the present embodiment is effective to provide a beneficial change in the subject's 24-hour pattern of SBP and/or DBP. The kinds of beneficial change that can be provided are more fully described hereinbelow.

Because of the particular criticality of controlling blood pressure in subjects with complicating conditions such as diabetes and/or chronic kidney disease, and the greater difficulty of lowering blood pressure to the lower levels consistent with good management of these conditions, the method of the present embodiment can be especially beneficial for such subjects.

In another embodiment of the invention, a method for lowering blood pressure in a subject exhibiting resistance to a baseline antihypertensive therapy with one or more drugs comprises administering to the subject, adjunctively with the baseline therapy, a compound of formula (I) above or a pharmaceutically acceptable salt thereof, wherein the baseline therapy is modified in a manner that results in a reduced risk or incidence of adverse events.

Examples of modifications of the baseline therapy which can result in a reduced risk or incidence of adverse events include, without limitation, dose reduction, elimination, split-dose administration, controlled release formulation, and/or selection of a non-peroral route of administration of at least one of the drugs in the baseline therapy. Another such modification comprises administering different drugs in the baseline therapy at different times of day instead of all at about the same time, a mode of administration termed “non-simultaneous” herein. The term “split-dose administration” means increasing the frequency of administration of a drug, for example from once to twice a day, without increasing the total daily dose of the drug.

Variants and illustrative modalities of the method of this embodiment, for example selection of a compound of formula (I), the baseline therapy employed, routes of administration, dosages, durations of treatment, frequency of administration, formulations of the compound of formula (I) and particular blood pressure benefits provided, are as described hereinabove.

In yet another embodiment of the invention, a method for lowering blood pressure in a subject exhibiting resistance to a baseline antihypertensive therapy with one or more drugs comprises administering to the subject, adjunctively with the baseline therapy, a compound of formula (I), wherein a beneficial change in the subject's 24-hour pattern of SBP and/or DBP is obtained. According to this embodiment, the adjunctively administered baseline therapy can be unmodified but is optionally modified (a) by dose reduction or elimination of at least one of said drugs and/or (b) in a manner that results in a reduced risk or incidence of adverse events.

The term “24-hour pattern” in relation to a blood pressure parameter such as SBP or DBP refers to a cycle in that parameter that recurs approximately daily, for example reflecting underlying endogenous circadian rhythms and/or blood levels of one or more drugs administered in an antihypertensive regimen. For example, increases, decreases, maxima and minima of blood pressure that typically occur each day or night around the same time or times are aspects of the 24-hour pattern. Further aspects include SBP or DBP measured at a specific time in relation to the timing of administration of an antihypertensive drug, for example a compound of formula (I). Illustratively, SBP or DBP measured shortly before the regular time of administration is referred to as “trough” SBP or DBP, being measured at a time when levels of the drug circulating in the bloodstream are assumed to be at their lowest. Thus, as explained above, where the drug is administered once daily at around 8 am, the trough SBP or DBP relates to a blood pressure measurement taken shortly before 8 am on any day. Blood pressure measurements can be recorded in a sitting or reclining subject. In one embodiment, however, 24-hour pattern and effects of an antihypertensive regimen thereon are established for an ambulatory subject by ABP monitoring.

Examples of beneficial changes in aspects of the 24-hour blood pressure pattern include without limitation
(a) lowering of 24-hour mean ABP;
(b) lowering of trough sitting SBP;
(c) lowering of trough sitting DBP;
(d) lowering of diurnal maximum ABP;
(e) trend away from a bimodal waveform pattern towards a unimodal or less pronouncedly bimodal pattern consistent with normotensive subjects;
(f) increase in day/night ABP ratio; and
(g) at least about 10% nocturnal dipping of ABP.

In one such aspect, the method of the present embodiment is effective to increase day/night ABP ratio, for example from a baseline below about 10% to greater than 10%. Day/night ABP ratio can illustratively be increased by at least about 2, for example at least about 3 or at least about 5 percentage points.

In another aspect, the method of the present embodiment is effective to lower blood pressure in all phases of a 24-hour blood pressure cycle, for example as measured by ABP monitoring at a suitable interval, e.g., hourly. According to this aspect, the 24-hour blood pressure cycle can exhibit a bimodal waveform pattern both at baseline and when treated with a compound of formula (I) according to the present method, but treatment with the compound of formula (I) in combination with the baseline therapy shifts the waveform pattern downward.

The method of the present embodiment optionally permits dose reduction or elimination of at least one of the drugs in the baseline therapy, and/or optionally results in a reduced risk or incidence of adverse events when the baseline therapy is modified as described above, by comparison with the unmodified baseline therapy alone.

Variants and illustrative modalities of the method of this embodiment, for example selection of a compound of formula (I), the baseline therapy employed, routes of administration, dosages, durations of treatment, frequency of administration, and formulations of the compound of formula (I) are as described hereinabove.

In the embodiments described above, the compound of formula (I) is administered adjunctively with a baseline antihypertensive therapy comprising administration of one or more additional drugs. Each of these additional drugs can be administered at a full, adequate or reduced dose. One of skill in the art can readily identify a suitable full dose for any particular drug mentioned here from publicly available information in printed or electronic form, for example on the internet, and can, if desired, reduce the dose in accordance with certain embodiments of the present invention based on the disclosure herein.

Mention of a particular diuretic or antihypertensive drug in the present specification and claims will be understood, except where the context demands otherwise, to include pharmaceutically acceptable salts, esters, prodrugs, metabolites, racemates and enantiomers of the drug, to the extent that such salts, esters, prodrugs, metabolites, racemates or enantiomers exist and are therapeutically effective.

Examples of drugs useful in combination or adjunctive therapy with a compound of formula (I) or as a component of a baseline antihypertensive therapy are classified and presented in several lists below. Some drugs are active at more than one target; accordingly certain drugs may appear in more than one list. Use of any listed drug in a combination or adjunctive therapy of the invention is contemplated herein, independently of its mode of action.

A suitable diuretic can illustratively be selected from the following list.

Organomercurials
chlormerodrin
meralluride
mercaptomerin sodium
mercumatilin sodium
mercurous chloride
mersalyl

Purines
pamabrom
protheobromine
theobromine

Steroids
canrenone
eplerenone
oleandrin
spironolactone

Sulfonamide Derivatives
acetazolamide
ambuside
azosemide
bumetanide
butazolamide
chloraminophenamide
clofenamide
clopamide
clorexolone
disulfamide
ethoxzolamide
furosemide
mefruside
methazolamide
piretamide
torsemide
tripamide
xipamide

Thiazides and Analogs
althiazide
bendroflumethiazide
benzthiazide
benzylhydrochlorothiazide
buthiazide
chlorothiazide
chlorthalidone
cyclopenthiazide
cyclothiazide
ethiazide
fenquizone
hydrochlorothiazide
hydroflumethiazide
indapamide
methyclothiazide
metolazone
paraflutizide
polythiazide
quinethazone
teclothiazide
trichlormethiazide

Uracils
aminometradine

Unclassified
amiloride
Biogen BG 9719
chlorazanil
ethacrynic acid
etozolin
isosorbide
Kiowa Hakko KW 3902
mannitol
muzolimine
perhexiline
Sanofi-Aventis SR 121463
ticrynafen
triamterene
urea

In some embodiments, the diuretic comprises a thiazide or loop diuretic. Thiazide diuretics are generally not preferred where the subject has a complicating condition such as diabetes or chronic kidney disease, and in such situations a loop diuretic can be a better choice.

Particularly suitable thiazide diuretics include chlorothiazide, chlorthalidone, hydrochlorothiazide, indapamide, metolazone, polythiazide and combinations thereof. Particularly suitable loop diuretics include bumetanide, furosemide, torsemide and combinations thereof.

A suitable ACE inhibitor can illustratively be selected from the following list:
alacepril
benazepril
captopril
ceronapril
cilazapril
delapril
enalapril
enalaprilat
eosinopril
fosinopril
imidapril
lisinopril
moexipril
moveltipril
omapatrilat
perindopril
quinapril
ramipril
sampatrilat
spirapril
temocapril
trandolapril

Particularly suitable ACE inhibitors include benazepril, captopril, enalapril, fosinopril, lisinopril, moexipril, perindopril, quinapril, ramipril, trandolapril and combinations thereof.

A suitable angiotensin II receptor blocker can illustratively be selected from the following list:
candesartan
eprosartan
irbesartan
losartan
olmesartan
tasosartan
telmisartan
valsartan

A suitable beta-adrenergic receptor blocker can illustratively be selected from the following list:
AC 623
acebutolol
alprenolol
atenolol
amosulalol
arotinolol
atenolol
befunolol
betaxolol
bevantolol
bisoprolol
bopindolol
bucindolol
bucumolol
bufetolol
bufuralol
bunitrolol
bupranolol
butidrine hydrochloride
butofilolol
carazolol
carteolol
carvedilol
celiprolol
cetamolol
cloranolol
dilevalol
esmolol
indenolol
labetalol
landiolol
levobunolol
mepindolol
metipranolol
metoprolol
moprolol
nadolol
nadoxolol
nebivolol
nifenalol
nipradilol
oxprenolol
penbutolol
pindolol
practolol
pronethalol
propranolol
sotalol
sulfinalol
talinolol
tertatolol
tilisolol
timolol
toliprolol
xibenolol

Particularly suitable beta-adrenergic receptor blockers include acebutolol, atenolol, betaxolol, bisoprolol, carvedilol, labetalol, metoprolol, nadolol, penbutolol, pindolol, propranolol, timolol and combinations thereof.

A suitable calcium channel blocker can illustratively be selected from the following list:

Aryklalkylamines
bepridil
clentiazem
diltiazem
fendiline
gallopamil
mibefradil
prenylamine
semotiadil
terodiline
verapamil

Dihydropyridine Derivatives
amlodipine
aranidipine
barnidipine
benidipine
cilnidipine
efonidipine
elgodipine
felodipine
isradipine
lacidipine
lercanidipine
manidipine
nicardipine
nifedipine
nilvadipine
nimodipine
nisoldipine
nitrendipine
NZ 105

Piperazine Derivatives
cinnarizine
dotarizine
flunarizine
lidoflazine
lomerizine

Unclassified
bencyclane
etafenone
fantofarone
monatepil
perhexiline

Particularly suitable calcium channel blockers include amlodipine, diltiazem, felodipine, isradipine, nicardipine, nifedipine, nisoldipine, verapamil and combinations thereof.

A suitable direct vasodilator can illustratively be selected from the following list:
amotriphene
benfurodil hemisuccinate
benziodarone
chloracizine
chromonar
clobenfurol
clonitrate
cloricromen
dilazep
droprenilamine
efloxate
erythrityl tetranitrate
etafenone
fendiline
hexestrol bis(β-diethylaminoethyl ether)
hexobendine
hydralazine
isosorbide dinitrate
isosorbide mononitrate
itramin tosylate
khellin
lidoflazine
mannitol hexanitrate
minoxidil
nitroglycerin
pentaerythritol tetranitrate
pentrinitrol
perhexiline
pimefylline
prenylamine
propatyl nitrate
trapidil
tricromyl
trimetazidine
trolnitrate phosphate
visnadine

Particularly suitable direct vasodilators include hydralazine, minoxidil and combinations thereof.

A suitable alpha-1-adrenergic receptor blocker can illustratively be selected from the following list:
amosulalol
arotinolol
carvedilol
dapiprazole
doxazosin
ergoloid mesylates
fenspiride
idazoxan
indoramin
labetalol
methyldopa
monatepil
naftopidil
nicergoline
prazosin
tamsulosin
terazosin
tolazoline
trimazosin
yohimbine

Particularly suitable alpha-1-adrenergic receptor blockers include carvedilol, doxazosin, labetalol, prazosin, terazosin and combinations thereof. It is noted that, of these, carvedilol and labetalol also function as beta-adrenergic receptor blockers.

A suitable central alpha-2-adrenergic receptor agonist or other centrally acting antihypertensive drug can illustratively be selected from the following list:
clonidine
guanabenz
guanadrel
guanfacine
methyldopa
moxonidine
reserpine

A suitable aldosterone receptor antagonist can illustratively be selected from the following list:
canrenone
eplerenone
spironolactone

Illustrative Vasopeptidase Inhibitors Include:
fasidotril
omapatrilat
sampatrilat

Illustrative NEP Inhibitors, Some of which are Also ACE Inhibitors, Include:
candoxatril
CGS 26582
MDL 100173
omapatrilat
phosphoramidon
sinorphan
thiorphan
Z13752A

Illustrative Prostanoids Include:
beraprost
cicaprost
epoprostenol
iloprost
PGE₁
PGI₂ (prostacyclin)
NS-304
treprostinil

Illustrative PDE5 Inhibitors Include:
sildenafil
tadalafil
vardenafil

Inhibitors of Renin Activity or Release Include Renin Inhibitors, Illustratively:
aliskiren
ciprokiren
ditekiren
enalkiren
remikiren
terlakiren
zankiren

The term “renin inhibitor” herein means an inhibitor of the enzymatic activity of renin. A particularly suitable renin inhibitor is aliskiren.

Other drugs that can be useful in combination or adjunctive therapy with a compound of formula (I) or in a baseline antihypertensive therapy can illustratively be selected from the following unclassified list:
ajmaline
alfuzosin
Alteon ALT 711
γ-aminobutyric acid
atrial natriuretic peptide
azelnidipine
bethanidine
bietaserpine
bosentan
budralazine
bufeniode
bunazosin
cadralazine
carmoxirole
CD 3400
chlorisondamine chloride
cicletanine
ciclosidomine
clevidipine
debrisoquin
denitronipradilol
desacetylalacepril
deserpidine
diazoxide
dihydralazine
endralazine
fenoldopam
flosequinan
guanethidine
guanidine, N-cyano-N′-4-pyridinyl-N″-(1,2,2-trimethylpropyl)-, monohydrate
guanoxabenz
guanoxan
hexamethonium
ketanserin
LBI 45
levcromakalim
lofexidine
magnesiocard
mebutamate
mecamylamine
normopresil
2-oxazolamine, N-(dicyclopropylmethyl)-4,5-dihydro-, (2E)-2-butenedioate
pargyline
pempidine
pentamethonium bromide
pentolinium tartrate
pheniprazine
phentolamine
pildralazine
pinacidil
piperoxan
protoveratrines
3,5-pyridinedicarboxylic acid, 1,4-dihydro-2,6-dimethyl-4-(3-nitrophenyl)-,
methyl 1-(phenylmethyl)-3-pyrrolidinyl ester
raubasine
rescimetol
rescinnamine
rilmenidine
saralasin
sodium nitroprusside
syrosingopine
Takeda TAK 536
tetrahydrolipstatin
1,4-thiazepine-4(5H)-acetic acid, 6-[[1-(ethoxycarbonyl)-3-phenylpropyl]-amino]tetrahydro-5-oxo-2-(2-thienyl)
tiamenidine
todralazine
tolonidine
trimethaphan camsylate
tyrosinase
urapidil
zofenopril

In one embodiment, the compound of formula (I) is administered concomitantly (e.g., in combination or adjunctive therapy) with one or more of
(a) a diuretic selected from the group consisting of chlorothiazide, chlorthalidone, hydrochlorothiazide, indapamide, metolazone, polythiazide, bumetanide, furosemide, torsemide and combinations thereof;
(b) an ACE inhibitor selected from the group consisting of benazepril, captopril, enalapril, fosinopril, lisinopril, moexipril, perindopril, quinapril, ramipril, trandolapril and combinations thereof, and/or an angiotensin II receptor blocker selected from the group consisting of candesartan, eprosartan, irbesartan, losartan, olmesartan, tasosartan, telmisartan, valsartan and combinations thereof;
(c) a beta-adrenergic receptor blocker selected from the group consisting of acebutolol, atenolol, betaxolol, bisoprolol, carvedilol, labetalol, metoprolol, nadolol, penbutolol, pindolol, propranolol, timolol and combinations thereof;
(d) a calcium channel blocker selected from the group consisting of amlodipine, diltiazem, felodipine, isradipine, nicardipine, nifedipine, nisoldipine, verapamil and combinations thereof;
(e) a direct vasodilator selected from the group consisting of hydralazine, minoxidil and combinations thereof,
(f) an alpha-1-adrenergic receptor blocker selected from the group consisting of carvedilol, doxazosin, labetalol, prazosin, terazosin and combinations thereof;
(g) a central alpha-2-adrenergic receptor agonist or other centrally acting drug selected from the group consisting of clonidine, guanabenz, guanadrel, guanfacine, methyldopa, moxonidine, reserpine and combinations thereof;
(h) an aldosterone receptor antagonist selected from the group consisting of canrenone, eplerenone, spironolactone and combinations thereof; and
(i) the renin inhibitor aliskiren.

More particularly, the compound of formula (I) can be administered in combination or adjunctive therapy with one or more of (a), (b), (c) and (d) above, optionally further with one or more of (e), (f), (g), (h) and (i).

Still more particularly, the compound of formula (I) can be administered in combination or adjunctive therapy with at least (a) and any two of (b), (c) and (d).

The compound of formula (I), and the one or more drugs constituting the baseline antihypertensive therapy and optionally administered in combination with the compound of formula (I) can be delivered by any suitable route of administration. Orally bioavailable drugs are particularly suitable, in particular those that are suitable for once a day oral administration. Thus in one embodiment at least one of the diuretic or antihypertensive drugs in the baseline therapy is orally administered once a day. In a particular embodiment, all drugs in the baseline therapy are orally administered once a day. According to this embodiment, it will generally be found convenient to administer all drugs in the regimen, i.e., the compound of formula (I) as well as the baseline therapy drugs, orally once a day.

When a compound of formula (I) is used in adjunctive therapy with one or more baseline drugs, the compound of formula (I) and at least one baseline drug can be administered at different times or at about the same time (at exactly the same time or directly one after the other in any order). The compound of formula (I) and the at least one baseline drug can be formulated in one dosage form as a fixed-dose combination for administration at the same time, or in two or more separate dosage forms for administration at the same or different times.

Fixed-dose combinations of two or more drugs can be achieved in many cases by coformulation of the drugs in a single dosage unit such as a tablet or capsule. For example, coformulations of various drugs useful in a baseline antihypertensive therapy as defined herein are available, including:
amiloride+hydrochlorothiazide;
amlodipine+benazepril;
atenolol+chlorthalidone;
benazepril+hydrochlorothiazide;
bisoprolol+hydrochlorothiazide;
candesartan+hydrochlorothiazide;
captopril+hydrochlorothiazide;
enalapril+felodipine;
enalapril+hydrochlorothiazide;
eprosartan+hydrochlorothiazide;
fosinopril+hydrochlorothiazide;
irbesartan+hydrochlorothiazide;
lisinopril+hydrochlorothiazide;
losartan+hydrochlorothiazide;
methyldopa+hydrochlorothiazide;
metoprolol+hydrochlorothiazide;
moexipril+hydrochlorothiazide;
nadolol+hydrochlorothiazide;
olmesartan+hydrochlorothiazide;
propranolol+hydrochlorothiazide;
quinapril+hydrochlorothiazide;
reserpine+chlorothiazide;
reserpine+chlorthalidone;
reserpine+hydrochlorothiazide;
spironolactone+hydrochlorothiazide;
telmisartan+hydrochlorothiazide;
timolol+hydrochlorothiazide;
trandolapril+verapamil;
triamterene+hydrochlorothiazide; and
valsartan+hydrochlorothiazide.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, a compound of formula (I) and at least one drug useful in combination or adjunctive therapy with the compound of formula (I). In another example, the compound of formula (I) and the at least one drug useful in combination or adjunctive therapy with the compound of formula (I) are separately packaged and available for sale independently of one another, but are co-marketed or co-promoted for use according to the invention. The separate dosage forms can also be presented to a subject separately and independently, for use according to the invention.

In an embodiment of the invention, a method is provided for treating a hypertensive disorder, comprising administering in combination therapy a compound of formula (I), more particularly TBC3711, and at least one inhibitor of renin activity or release, for example a renin inhibitor such as aliskiren, ciprokiren, ditekiren, enalkiren, remikiren, terlakiren or zankiren.

Examples of hypertensive disorders that can be treated by the method of this embodiment include conditions marked by systolic hypertension, diastolic hypertension or both, including isolated systolic hypertension and hypertension in the elderly; such conditions can be primary (essential hypertension) or secondary to other conditions including obesity, diabetes, renal disorders (e.g., chronic renal failure, renovascular disease, diabetic nephropathy, etc.), adrenal disorders (e.g., adrenocortical and mineralocorticoid hypertension, pheochromocytoma, primary aldosteronism, Cushing's syndrome, etc.), insulin resistance, salt-sensitivity, polycystic ovary syndrome, sleep apnea, preeclampsia, thyroid and parathyroid diseases, and transplantation. Whether primary or secondary, such hypertension can be, as described above, resistant to baseline antihypertensive therapies, including resistant hypertension as clinically defined or diagnosed. Hypertensive disorders also include pulmonary arterial hypertension, which likewise can be primary or secondary to various conditions including diseases of the scleroderma spectrum (e.g., mixed connective tissue disease, Raynaud's disease, CREST syndrome, systemic sclerosis, or overlap syndrome); rheumatoid arthritis; chronic hepatitis; systemic lupus erythematosus; anorexigen use; human immunodeficiency virus (HIV) infection; chronic hypoxemia resulting from conditions such as chronic bronchitis, emphysema, sleep apnea, interstitial lung disease, or pulmonary fibrosis; thromboembolic diseases such as in situ thrombosis, tumors, or sickle cell disease; volume and pressure overloads induced primarily from disorders of the left heart (for example, chronic heart failure, septal defects, mitral valve disease, and left atrial myxoma); and disorders directly affecting the pulmonary vasculature such as schistosomiasis, sarcoidosis and pulmonary capillary hemangiomatosis.

The method can be used, for example, to lower blood pressure in a subject exhibiting resistance to a baseline antihypertensive therapy as described above, for example a subject having resistant hypertension. Optionally, administration of the compound of formula (I) and the inhibitor of renin activity or release is adjunctive to the baseline therapy, which can, if desired, be modified as described above, for example by dose reduction or elimination of at least one of the drugs in the baseline therapy.

Variants and illustrative modalities of the method of this embodiment, for example selection of a compound of formula (I), routes of administration, dosages, durations of treatment, frequency of administration, formulations of the compound of formula (I) and particular blood pressure benefits provided, are as described hereinabove.

In a further embodiment, a method for lowering blood pressure in a subject having diabetes and/or chronic kidney disease comprises administering a compound of formula (I) to the subject. The particular effectiveness of compounds of formula (I) in lowering blood pressure is believed to be especially useful in such a subject, given the criticality of blood pressure control and the more aggressive SBP and DBP goals (per JNC 7, <130 mmHg and <80 mmHg respectively) in such a subject. The subject can be, but is not necessarily, one exhibiting resistance to a baseline antihypertensive therapy, for example a subject having resistant hypertension. A monotherapy or combination or adjunctive therapy of a compound of formula (I) with one or more additional drug(s) as described herein can be administered.

For example, such additional drug(s) can comprise a diuretic and/or one or more antihypertensive drugs selected from the group consisting of (a) ACE inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) vasopeptidase inhibitors, (i) NEP inhibitors, (j) prostanoids, (k) PDE5 inhibitors, (l) nitrosylated compounds, (m) oral nitrates and (n) inhibitors of renin activity or release.

Variants and illustrative modalities of the present embodiment, for example selection of a compound of formula (I), routes of administration, dosages, durations of treatment, frequency of administration, formulations of the compound of formula (I) and particular blood pressure benefits provided, are as described hereinabove.

In a still further embodiment, a method for providing a beneficial effect on renal and/or cardiovascular function in a subject having resistant hypertension comprises administering a compound of formula (I) to the subject. “Providing a beneficial effect” in the present context includes enhancing, maintaining or moderating a decline in renal or cardiovascular function and also includes preventing one or more cardiovascular adverse events. A monotherapy or combination or adjunctive therapy of a compound of formula (I) with one or more additional drugs as described herein can be administered.

In one aspect of this embodiment, a method for preventing one or more cardiovascular adverse events in a subject having resistant hypertension comprises administering a compound of formula (I) to the subject. Examples of cardiovascular adverse effects include without limitation acute coronary syndrome (including unstable angina and non-Q wave infarction), myocardial infarction, heart failure, systolic heart failure, diastolic heart failure (also known as diastolic dysfunction), stroke, occlusive stroke, hemorrhagic stroke and combinations thereof. “Preventing” in the present context includes reducing risk, incidence and/or severity of a subsequent cardiovascular adverse effect. A monotherapy or combination or adjunctive therapy of a compound of formula (I) with one or more additional drugs as described herein can be administered.

In another aspect of the present embodiment, a method for providing a beneficial effect on renal function in a subject having resistant hypertension comprises administering a compound of formula (I) to the subject. A monotherapy or combination or adjunctive therapy of a compound of formula (I) with one or more additional drugs as described herein can be administered.

A beneficial effect on renal function can be observed, for example, by monitoring one or more blood and/or urinary biomarkers. Examples of such biomarkers include without limitation serum creatinine, serum insulin, serum glutamic acid decarboxylase (GAD), serum protein tyrosine phosphatase-like molecule IA2, blood urea nitrogen, urinary protein, urinary albumin, microalbuminuria, urinary β2-microglobulin, urinary N-acetyl-β-glucosaminidase, urinary retinol binding protein, urinary sodium, glomerular filtration rate, urinary albumin to creatinine ratio, urine volume, and combinations thereof.

Illustratively, the compound of formula (I) can be administered in a dose effective to lower urinary albumin to creatinine ratio. This can be especially beneficial where the baseline urinary albumin to creatinine ratio is greater than about 30 mg/g or where baseline 24-hour urinary albumin is greater than about 30 mg/day.

Variants and illustrative modalities of the method of the present embodiment, for example selection of a compound of formula (I), routes of administration, dosages, durations of treatment, frequency of administration, formulations of the compound of formula (I), patient population and particular blood pressure benefits provided, are as described hereinabove.

A therapeutic combination comprising a compound of formula (I), at least one diuretic, and at least two antihypertensive drugs selected from at least two of (a) ACE inhibitors and angiotensin II receptor blockers, (b) beta-adrenergic receptor blockers and (c) calcium channel blockers, wherein the at least one diuretic and/or the at least one antihypertensive drug are present at substantially less than a full dose, is itself a further embodiment of the invention. “Substantially less than a full dose” is as defined and illustrated hereinabove.

Such a combination can have utility in a number of situations, not limited to methods described herein. However, a combination of this embodiment can be especially useful for lowering blood pressure in a subject exhibiting resistance to a baseline antihypertensive therapy with one or more drugs; for lowering blood pressure in a subject having diabetes and/or chronic kidney disease; and/or for producing a beneficial effect on renal and/or cardiovascular function in a subject having resistant hypertension.

The at least one diuretic in the combination can illustratively be selected from those listed hereinabove. In particular embodiments the diuretic comprises a thiazide diuretic or a loop diuretic. Suitable ACE inhibitors, angiotensin II receptor blockers, beta-adrenergic receptor blockers and calcium channel blockers can illustratively be selected from those listed hereinabove. Optionally, the combination can further comprise one or more additional drugs selected from direct vasodilators, alpha-1-receptor blockers, central alpha-2-adrenergic receptor agonists and other centrally acting antihypertensive drugs, and aldosterone receptor antagonists. Suitable drugs of these classes are illustratively listed hereinabove.

In one embodiment, the combination comprises a compound of formula (I), for example sitaxsentan or TBC3711, plus (a) and at least two of (b), (c) and (d) as described below:
(a) a diuretic selected from the group consisting of chlorothiazide, chlorthalidone, hydrochlorothiazide, indapamide, metolazone, polythiazide, bumetanide, furosemide, torsemide and combinations thereof;
(b) an angiotensin converting enzyme inhibitor selected from the group consisting of benazepril, captopril, enalapril, fosinopril, lisinopril, moexipril, perindopril, quinapril, ramipril, trandolapril and combinations thereof, and/or an angiotensin II receptor blocker selected from the group consisting of candesartan, eprosartan, irbesartan, losartan, olmesartan, tasosartan, telmisartan, valsartan and combinations thereof;
(c) a beta-adrenergic receptor blocker selected from the group consisting of acebutolol, atenolol, betaxolol, bisoprolol, carvedilol, labetalol, metoprolol, nadolol, penbutolol, pindolol, propranolol, timolol and combinations thereof;
(d) a calcium channel blocker selected from the group consisting of amlodipine, diltiazem, felodipine, isradipine, nicardipine, nifedipine, nisoldipine, verapamil and combinations thereof.

In another embodiment, a therapeutic combination is provided, comprising a compound of formula (I), for example sitaxsentan or TBC3711, and an inhibitor of renin activity or release, for example a renin inhibitor such as aliskiren, ciprokiren, diteidren, enalkiren, remikiren, terlakiren or zankiren.

Typically in a therapeutic combination of the invention, at least the compound of formula (I) is provided in an orally deliverable formulation, for example a formulation adapted for oral delivery of a compound of formula (I) dose of about 1 to about 600 mg/day, e.g., about 10 to about 200 mg/day. The formulation can be adapted for any suitable frequency of administration, but in one embodiment is adapted for once a day oral administration.

In one embodiment at least one of the drugs other than the compound of formula (I) in the combination is provided in an orally deliverable formulation; for example, each of the drugs can be so provided, and each of the drugs can be in a formulation adapted for once a day oral administration. Each of the drugs other than the compound of formula (I) can be present in the combination in an amount providing a full, adequate or reduced dose of the drug as indicated hereinabove. One of skill in the art can readily identify a full dose for any particular drug from publicly available information in printed or electronic form, for example on the internet, and can, if desired, reduce the dose in accordance with certain embodiments of the present invention based on the disclosure herein.

Any two or more drugs in the combination can optionally be coformulated to provide a fixed dose combination. For example, the compound of formula (I) can be coformulated with any one or more of the other drugs in the combination.

All patents and publications cited herein are incorporated by reference into this application in their entirety.

The words “comprise”, “comprises”, and “comprising” are to be interpreted inclusively rather than exclusively.

What is claimed is:

1. A method for lowering blood pressure in a subject exhibiting resistance to a baseline antihypertensive therapy with one or more drugs, the method comprising administering to the subject, adjunctively with the baseline therapy, a compound of formula (I) 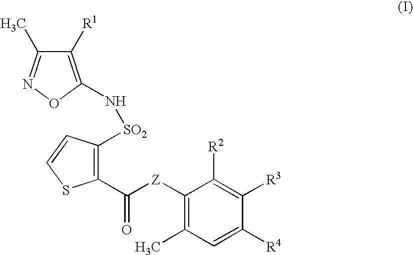
or a pharmaceutically acceptable salt thereof, wherein:
R¹ is halo or C₁₋₃ alkyl;
R² is hydrogen, C₁₋₃ alkyl, (C₁₋₃ alkyl)carbonyl, (C₃₋₆ cycloalkyl)carbonyl, cyano, halo, aminocarbonyl, di(C₁₋₃ alkyl)aminocarbonyl, (C₁₋₃ alkyl)sulfonyl, oxazol-2-yl or benzoyl;
R³ and R⁴ are independently hydrogen or C₁₋₀₃ alkyl, or together form a methylenedioxy bridge; and
Z is CH₂or NH;
and wherein
(a) the adjunctively administered baseline therapy is modified by dose reduction or elimination of at least one of said drugs;
(b) the adjunctively administered baseline therapy is modified in a manner that results in a reduced risk or incidence of adverse events; and/or
(c) a beneficial change in the subject's 24-hour pattern of systolic and/or diastolic blood pressure is obtained.

2. The method of claim 1, wherein the subject has diabetes, chronic kidney disease or both.

3. The method of claim 1, wherein the compound of formula (I) is sitaxsentan or TBC3711.

4. The method of claim 3, wherein the compound of formula (I) is TBC3711 and is administered in a daily dose of about 1 to about 600 mg.

5. The method of claim 1, wherein the baseline therapy to which the subject exhibits resistance comprises administration of one or more diuretics and/or one or more antihypertensive drugs selected from the group consisting of (a) ACE inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) vasopeptidase inhibitors, (i) NEP inhibitors, (j) prostanoids, (k) PDE5 inhibitors, (l) nitrosylated compounds, (m) oral nitrates and (n) inhibitors of renin activity or release.

6. The method of claim 1, wherein the subject has resistant hypertension, and the baseline therapy to which the subject exhibits resistance comprises administration of at least one diuretic and at least two antihypertensive drugs selected from at least two of (a) ACE inhibitors and angiotensin II receptor blockers, (b) beta-adrenergic receptor blockers and (c) calcium channel blockers.

7. The method of claim 1, wherein the adjunctively administered baseline therapy is modified by dose reduction or elimination of at least one of said drugs and/or in a manner that results in a reduced risk or incidence of adverse events.

8. The method of claim 7, wherein the compound of formula (I) is administered at a dose and frequency effective, in combination with the modified baseline therapy, to provide a reduction of at least about 3 mmHg in one or more blood pressure parameters selected from trough sitting systolic, trough sitting diastolic, 24-hour ambulatory systolic, 24-hour ambulatory diastolic, maximum diurnal systolic and maximum diurnal diastolic blood pressures.

9. The method of claim 8, wherein the subject has resistant systolic hypertension, and the compound of formula (I) is administered at a dose and frequency effective, in combination with the modified baseline therapy, to provide a reduction of at least about 3 mmHg in one or more systolic blood pressure parameters selected from trough sitting, 24-hour ambulatory and maximum diurnal systolic blood pressures.

10. The method of claim 9, wherein a JNC 7, BHD-IV, ESH/ESC or WHO/ISH goal for systolic blood pressure is achieved.

11. The method of claim 8, wherein the subject has resistant diastolic hypertension, and the compound of formula (I) is administered at a dose and frequency effective, in combination with the modified baseline therapy, to provide a reduction of at least about 3 mmHg in one or more diastolic blood pressure parameters selected from trough sitting, 24-hour ambulatory and maximum diurnal diastolic blood pressures.

12. The method of claim 11, wherein a JNC 7, BHD-IV, ESH/ESC or WHO/ISH goal for diastolic blood pressure is achieved.

13. The method of claim 7, wherein the adjunctively administered baseline therapy is modified by dose reduction or elimination, by split-dose administration, by non-simultaneous administration of different drugs, by controlled release formulation and/or by selection of a non-peroral route of administration of at least one of said drugs.

14. The method of claim 1, wherein a beneficial change in the subject's 24-hour pattern of systolic and/or diastolic blood pressure is obtained and the adjunctively administered baseline therapy is optionally modified (a) by dose reduction or elimination of at least one of said drugs and/or (b) in a manner that results in a reduced risk or incidence of adverse events.

15. The method of claim 14, wherein the subject at baseline exhibits a bimodal waveform 24-hour pattern of systolic and/or diastolic blood pressure.

16. The method of claim 14, wherein the beneficial change comprises at least one of
(a) lowering of 24-hour mean ambulatory blood pressure;
(b) lowering of trough sitting systolic blood pressure;
(c) lowering of trough sitting diastolic blood pressure;
(d) lowering of diurnal maximum ambulatory blood pressure;
(e) a trend away from a bimodal waveform pattern towards a unimodal or less pronouncedly bimodal pattern consistent with normotensive subjects;
(f) an increase in day/night ambulatory blood pressure ratio; and
(g) at least about 10% nocturnal dipping of ambulatory blood pressure.

17. The method of claim 14, wherein the beneficial change is evident from ambulatory blood pressure monitoring.

18. The method of claim 14, wherein the subject has resistant systolic hypertension, and the compound of formula (I) is administered at a dose and frequency effective, in combination with the optionally modified baseline therapy, to provide a reduction of at least about 3 mmHg in one or more systolic blood pressure parameters selected from trough sitting, 24-hour ambulatory and maximum diurnal systolic blood pressures.

19. The method of claim 18, wherein a JNC 7, BHD-IV, ESH/ESC or WHO/ISH goal for systolic blood pressure is achieved.

20. The method of claim 14, wherein the subject has resistant diastolic hypertension, and the compound of formula (I) is administered at a dose and frequency effective, in combination with the optionally modified baseline therapy, to provide a reduction of at least about 3 mmHg in one or more diastolic blood pressure parameters selected from trough sitting, 24-hour ambulatory and maximum diurnal diastolic blood pressures.

21. The method of claim 20, wherein a JNC 7, BHD-IV, ESH/ESC or WHO/ISH goal for diastolic blood pressure is achieved.

22. A method for treating a hypertensive disorder, comprising administering in combination therapy TBC3711 and at least one inhibitor of renin activity or release.

23. The method of claim 22, wherein the hypertensive disorder is selected from the group consisting of systolic hypertension; diastolic hypertension; isolated systolic hypertension; hypertension in the elderly; essential hypertension; hypertension secondary to obesity, diabetes, renal disorders, adrenal disorders, Cushing's syndrome, insulin resistance, salt sensitivity, polycystic ovary syndrome, sleep apnea, preeclampsia, thyroid and parathyroid diseases and/or transplantation; and pulmonary arterial hypertension.

24. The method of claim 22, wherein the inhibitor of renin activity or release is a renin inhibitor selected from the group consisting of aliskiren, ciprokiren, ditekiren, enalkiren, remikiren, terlakiren and zankiren.

25. The method of claim 22, wherein the TBC3711 is administered in a daily dose of about 1 to about 600 mg.

26. A method for lowering blood pressure in a subject having diabetes and/or chronic kidney disease, the method comprising administering to the subject a compound of formula (I) 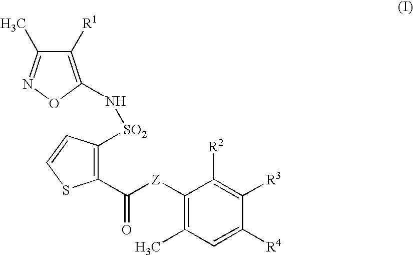
or a pharmaceutically acceptable salt thereof, wherein:
R¹ is halo or C₁₋₃ alkyl;
R² is hydrogen, C₁₋₃ alkyl, (C₁₋₃ alkyl)carbonyl, (C₃₋₆ cycloalkyl)carbonyl, cyano, halo, aminocarbonyl, di(C₁₋₃ alkyl)aminocarbonyl, (C₁₋₃ alkyl)sulfonyl, oxazol-2-yl or benzoyl;
R³ and R⁴ are independently hydrogen or C₁₋₃ alkyl, or together form a methylenedioxy bridge; and
Z is CH₂or NH.

27. The method of claim 26, wherein the compound of formula (I) is sitaxsentan or TBC3711.

28. The method of claim 26, wherein a blood pressure goal according to JNC 7, BHD-IV, ESH/ESC or WHO/ISH guidelines, more stringent than set for subjects not having diabetes or chronic kidney disease, is achieved.

29. The method of claim 26, wherein the subject exhibits resistance to a baseline antihypertensive therapy with one or more drugs, and the compound of formula (I) is administered adjunctively with said baseline therapy, which is optionally modified (a) by dose reduction or elimination of at least one of said drugs and/or (b) in a manner that results in a reduced risk or incidence of adverse events.

30. The method of claim 29, wherein the compound of formula (I) is administered at a dose and frequency effective, in combination with the optionally modified baseline therapy, to provide a reduction of at least about 3 mmHg in one or more blood pressure parameters selected from trough sitting systolic, trough sitting diastolic, 24-hour ambulatory systolic, 24-hour ambulatory diastolic, maximum diurnal systolic and maximum diurnal diastolic blood pressures.

31. The method of claim 29, wherein the baseline therapy comprises administration of one or more diuretics and/or one or more antihypertensive drugs selected from the group consisting of (a) ACE inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) vasopeptidase inhibitors, (i) NEP inhibitors, (j) prostanoids, (k) PDE5 inhibitors, (l) nitrosylated compounds, (m) oral nitrates and (n) inhibitors of renin activity or release.

32. The method of claim 29, wherein the subject has resistant hypertension, and the baseline therapy to which the subject exhibits resistance comprises administration of at least one diuretic and at least two antihypertensive drugs selected from at least two of (a) ACE inhibitors and angiotensin II receptor blockers, (b) beta-adrenergic receptor blockers and (c) calcium channel blockers.

33. The method of claim 32, wherein the subject has resistant systolic hypertension, and the compound of formula (I) is administered at a dose and frequency effective, in combination with the optionally modified baseline therapy, to provide a reduction of at least about 3 mmHg in one or more systolic blood pressure parameters selected from trough sitting, 24-hour ambulatory and maximum diurnal systolic blood pressures.

34. The method of claim 32, wherein the subject has resistant diastolic hypertension, and the compound of formula (I) is administered at a dose and frequency effective, in combination with the optionally modified baseline therapy, to provide a reduction of at least about 3 mmHg in one or more diastolic blood pressure parameters selected from trough sitting, 24-hour ambulatory and maximum diurnal diastolic blood pressures.

35. The method of claim 26, wherein the compound of formula (I) is TBC3711 and is administered in a daily dose of about 1 to about 600 mg.

36. A method for providing a beneficial effect on renal and/or cardiovascular function in a subject having resistant hypertension, the method comprising administering to the subject a compound of formula (I) 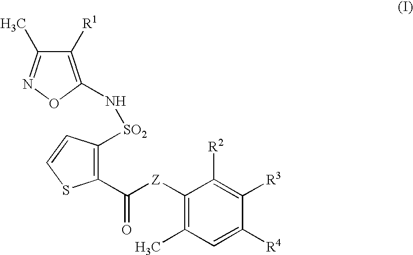
or a pharmaceutically acceptable salt thereof, wherein:
R¹ is halo or C₁₋₃ alkyl;
R² is hydrogen, C₁₋₃ alkyl, (C₁₋₃ alkyl)carbonyl, (C₃₋₆ cycloalkyl)carbonyl, cyano, halo, aminocarbonyl, di(C₁₋₃ alkyl)aminocarbonyl, (C₁₋₃ alkyl)sulfonyl, oxazol-2-yl or benzoyl;
R³ and R⁴ are independently hydrogen or C₁₋₃ alkyl, or together form a methylenedioxy bridge; and
Z is CH₂ or NH.

37. The method of claim 36, wherein the compound of formula (I) is sitaxsentan or TBC3711.

38. The method of claim 36, wherein the beneficial effect comprises preventing one or more cardiovascular adverse events in the subject.

39. The method of claim 38, wherein the one or more cardiovascular adverse events are selected from the group consisting of acute coronary syndrome, myocardial infarction, heart failure, systolic heart failure, diastolic heart failure, stroke, occlusive stroke, hemorrhagic stroke and combinations thereof.

40. The method of claim 36, wherein the beneficial effect is on renal function and is observable by monitoring one or more blood and/or urinary biomarkers.

41. The method of claim 40, wherein the one or more biomarkers are selected from the group consisting of serum creatinine, serum insulin, serum GAD, serum IA2, blood urea nitrogen, urinary protein, urinary albumin, microalbuminuria, urinary β2-microglobulin, urinary N-acetyl-β-glucosaminidase, urinary retinol binding protein, urinary sodium, glomerular filtration rate, urinary albumin to creatinine ratio, urine volume and combinations thereof.

42. The method of claim 40, wherein the compound of formula (I) is administered in a dose effective to lower urinary albumin to creatinine ratio.

43. The method of claim 42, wherein the subject exhibits, prior to administration of the compound of formula (I), a baseline urinary albumin to creatinine ratio greater than about 30 mg/g.

44. The method of claim 42, wherein the subject exhibits, prior to administration of the compound of formula (I), a baseline 24-hour urinary albumin greater than about 30 mg/day.

45. The method of claim 36, wherein the compound of formula (I) is administered adjunctively with one or more diuretics and/or one or more antihypertensive drugs selected from the group consisting of (a) ACE inhibitors and angiotensin II receptor blockers, (b) beta-adrenergic receptor blockers, (c) calcium channel blockers, (d) direct vasodilators, (e) alpha-1-adrenergic receptor blockers, (f) central alpha-2-adrenergic receptor agonists and other centrally acting antihypertensive drugs, (g) aldosterone receptor antagonists, (h) vasopeptidase inhibitors, (i) NEP inhibitors, (j) prostanoids, (k) PDE5 inhibitors, (l) nitrosylated compounds, (m) oral nitrates and (n) inhibitors of renin activity or release.

46. The method of claim 36, wherein the subject has resistant hypertension, and the compound of formula (I) is administered adjunctively with at least one diuretic and at least two antihypertensive drugs selected from at least two of (a) ACE inhibitors and angiotensin II receptor blockers, (b) beta-adrenergic receptor blockers and (c) calcium channel blockers.

47. The method of claim 36, wherein the subject has diabetes, chronic kidney disease or both.

48. The method of claim 36, wherein the compound of formula (I) is TBC3711 and is administered in a daily dose of about 1 to about 600 mg.

49. A therapeutic combination comprising (a) a compound of formula (I) 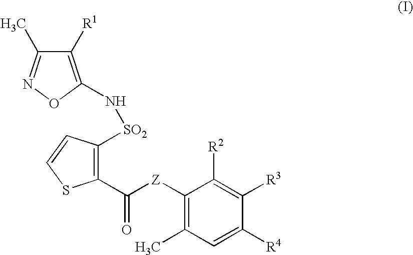
or a pharmaceutically acceptable salt thereof, wherein:
R¹ is halo or C₁₋₃ alkyl;
R² is hydrogen, C₁₋₃ alkyl, (C₁₋₃ alkyl)carbonyl, (C₃₋₆ cycloalkyl)carbonyl, cyano, halo, aminocarbonyl, di(C₁₋₃ alkyl)aminocarbonyl, (C₁₋₃ alkyl)sulfonyl, oxazol-2-yl or benzoyl;
R³ and R⁴ are independently hydrogen or C₁₋₃ alkyl, or together form a methylenedioxy bridge; and
Z is CH₂ or NH;
(b) at least one diuretic; and (c) at least one antihypertensive drug selected from the group consisting of ACE inhibitors, angiotensin II receptor blockers, beta-adrenergic receptor blockers and calcium channel blockers; wherein the at least one diuretic and/or the at least one antihypertensive drug are present at substantially less than a full dose.

50. The combination of claim 49, wherein the compound of formula (I) is sitaxsentan or TBC3711.

51. The combination of claim 49, comprising a compound of formula (I), at least one diuretic, and at least two antihypertensive drugs selected from at least two of (a) angiotensin converting enzyme inhibitors and angiotensin II receptor blockers, (b) beta-adrenergic receptor blockers and (c) calcium channel blockers, wherein the at least one diuretic and/or at least one of the antihypertensive drugs are present at substantially less than a full dose.

52. The combination of claim 49, further comprising one or more additional antihypertensive drugs.

53. The combination of claim 49, comprising a compound of formula (I); a diuretic selected from the group consisting of chlorothiazide, chlorthalidone, hydrochlorothiazide, indapamide, metolazone, polythiazide, bumetanide, furosemide, torsemide and combinations thereof; and at least two of
(a) an angiotensin converting enzyme inhibitor selected from the group consisting of benazepril, captopril, enalapril, fosinopril, lisinopril, moexipril, perindopril, quinapril, ramipril, trandolapril and combinations thereof, and/or an angiotensin II receptor blocker selected from the group consisting of candesartan, eprosartan, irbesartan, losartan, olmesartan, tasosartan, telmisartan, valsartan and combinations thereof;
(b) a beta-adrenergic receptor blocker selected from the group consisting of acebutolol, atenolol, betaxolol, bisoprolol, carvedilol, labetalol, metoprolol, nadolol, penbutolol, pindolol, propranolol, timolol and combinations thereof; and
(c) a calcium channel blocker selected from the group consisting of amlodipine, diltiazem, felodipine, isradipine, nicardipine, nifedipine, nisoldipine, verapamil and combinations thereof.

54. The combination of claim 49, wherein the compound of formula (I) is TBC3711 and is formulated in a form adapted for delivery of a TBC3711 dose of about 1 to about 600 mg/day.
